(19) United States
(12) Patent Application Publication
(10) Pub. No.: US 2023/0394317 A1
(43) Pub. Date: Dec. 7, 2023

(54) SYSTEM AND METHOD FOR TEXT MINING

(71) Applicant: The University of Melbourne, Victoria (AU)

(72) Inventors: Zenan ZHAI, Victoria (AU); Cornelia Maria VERSPOOR, Victoria (AU); Trevor Anthony COHN, Victoria (AU); Camilio THORNE, Frankfurt (DE); Saber AKHONDI, Amsterdam (NL); Christian DRUCKENBRODT, Frankfurt (DE)

(21) Appl. No.: 18/033,915

(22) PCT Filed: Nov. 1, 2021
(86) PCT No.: PCT/AU21/51282

§ 371 Date: Apr. 26, 2023

(30) Foreign Application Priority Data
Nov. 2, 2020 (AU) 2020903975

(51) Int. Cl. G06N 3/088; G06V 30/413; G06V 30/412; G06F 40/284; G06F 40/177; G06F 40/40; G06F 40/30; G06V 10/82; G06N 3/0442

(52) U.S. Cl. CPC G06N 3/088; G06V 30/413; G06V 30/412; G06F 40/284; G06N 3/0442; G06F 40/40; G06F 40/30; G06V 10/82; G06F 40/177

(57) ABSTRACT

A method for text mining from one or more tables is provided. The method includes the steps of: receiving one or more tables, the tables having one or more table labels, and one or more cells to be processed, transforming each of the cells into cell vector representations; encoding the one or more cell vector representations with a sequential 2D model; obtaining one or more table-level vector representations by summarising the semantics of the cell vector representations by an image classification model; and mapping the output of to an output vector which represents the probability of each of the table labels.

### TECHNICAL FIELD

The present invention relates to a system and method for text mining and in particular, categorizing and characterizing tables that contain text.

### BACKGROUND OF INVENTION

Table analysis is a problem that occurs in many contexts, for example in scientific publications and journal articles in science and economics. In evidence based medicine, summary data related to clinical trial populations often appears in tables or in a systematic review paper where key data elements of the reviewed studies will often be summarised in a table. In the chemical realm, many new chemical compounds which are discovered in commercial research are first disclosed via the patent system by way of patent specifications which contain details of the compound. Patent specifications may contain disclosures of new compounds in the form of tables within the patent specification. In practice, there can be a large number of tables presented in a patent specification and these tables can of very large size (up to >1,000 rows). Further, not all of the tables in the patent specification are relevant to the key findings in the patents.

Data extraction tools which parse text or the like exist, but very few of these tools are useful to extract information from tables. Further, tools that are able to process tables are limited since these tools are developed for processing web tables which are typically smaller and have a simpler structure compared to chemical compound tables in patent specifications.

These tools usually aim to categorize table by their structures (e.g. column-wise related or row-wise related), hence are not suitable to be adapted for analysing tables in patents based on their semantics.

Another issue is that patent specifications are usually not of satisfactory readability in terms of the ability to digest information in a document (which is typically a long document) to identify key information. Commercial chemical databases exist which provide more reliable and comprehensive data, but this is largely a manual process. As the number of new patent applications are increasing year on year, it becomes infeasible in terms of both time and budget to manually process all patent specifications.

It would be desirable to provide an automated tool for identifying the content of tables thereby assisting researchers to locate key information faster and more accurately. It would further be desirable to provide a method and system which ameliorates or at least alleviates one or more of the above-mentioned problems or provides a useful alternative.

A reference herein to a patent document or other matter which is given as prior art is not to be taken as an admission that that document or matter was known or that the information it contains was part of the common general knowledge as at the priority date of any of the claims.

### SUMMARY OF INVENTION

According to a first aspect, the present invention provides a method for text mining from one or more tables, the method including the steps of: (a) receiving one or more tables, the tables having one or more table labels, and one or more cells to be processed; (b) transforming each of the cells into cell vector representations; (c) encoding the one or more cell vector representations with a sequential 2-D model; (d) obtaining one or more table-level vector representations by summarising the semantics of the cell vector representations by an image classification model; and (e) mapping the output of step (d) to an output vector which represents the probability of each of the table labels.

Preferably the sequential 2D model includes one or more quad-directional long-short term memory network, and in particular Q-LSTM.

The method may further include the step of applying a machine learning paradigm to train a model from a labelled data set.

In an embodiment, a long-text transformer may be provided as the encoder. In another embodiment, pre-trained word vectors and character-level word representation may be provided as input to a LSTM-based encoder.

It will be appreciated that any suitable training of word embedding may be provided. For example, pre-trained word vectors may be provided and used as input which may be trained in advance using suitable domain-relevant data without need for manual labelling. In an alternative, it may trained de novo provided there are sufficient quantities of relevant data.

Preferably, the encoder (whether the long-text transformer is provided as the encoder or pre-trained word vectors and character-level word representation being provided as input to a LSTM-based encoder) is pre-trained with an in-domain dataset to achieve optimal performance. In the case of a long text-transformer (e.g. longformer, reformer, poolingformer) it may be pre-trained/fine-tuned. Otherwise, pre-trained word vectors (e.g. GLoVe, Word2Vec, Continuous Bag of Words CBOW) may be derived from in-domain datasets.

In the first aspect, namely a table-level classification method, the table level classification may include a table layout classification and/or the table level classification may include a table semantic classification.

Preferably, the method includes a step of pre-processing the one or more classified cells in each of the one or more tables to provide one or more pre-processed classified cells.

The pre-processing may be tokenisation by way of one or more of tools; e.g. OSCAR4, ChemTok, NBICGeneChemTokenizer, OpenNLP, CoreNLP, NLTK, spaCy Tokenizer and the like.

Preferably, the image classification is by way of a convolutional neural network such as one or more of ResNet18, VGG, DenseNet or Inception.

Preferably, the step of transforming each of the cells into cell vector representations includes utilising a long-text transformer or an LSTM-based embedder.

The method may include the step of utilising a transformer based language model, and generating contextualized word representations by combining the internal states of the model for use in Natural Language Processing (NLP) tasks.

The language model may be a long-text transformer encoder but may be one or more of BERT, ELMo, XLNet or Roberta. The language model BERT may be modified to accept tables. Preferably, the transformer being used can be determined by the size of tables. If the table contains less than 512 tokens, BERT, ELMo, XLNet, Roberta may be more preferable, otherwise the aforementioned long-text transformers are more preferable.

According to a second aspect the present invention provides a method for text mining from one or more tables, the method including the steps of: (a) receiving one or more tables, the tables having one or more cell labels, and one or more cells to be processed; (b) transforming each of the cells into cell vector representations; (c) encoding the one or more cell vector representations with a sequential 2-D model; and (d) for each cell, mapping the outputs of step (c) to an output vector which represents the probability of each of the cell labels.

Preferably, the sequential 2D model includes one or more quad-directional long-short term memory network and the sequential 2D model is Q-LSTM. The method may further include the step of: applying a machine learning paradigm to train a model from a labelled data set.

In the second aspect, namely a cell-level classification method, the model architecture may differ from the first aspect (tables) however an image classification model is not necessarily needed to summarize the table. Since cell-level classification is only being carried out, the vector representation of each cell can be directly mapped to the probability distribution over labels for each cell.

### BRIEF DESCRIPTION OF DRAWINGS

The invention will now be described in further detail by reference to the accompanying drawings. It is to be understood that the particularity of the drawings does not superseded the generality of the preceding description of the invention.

FIG. 1 is a schematic diagram of an example network that can be utilised to give effect to the system according to an embodiment of the invention;

FIG. 2a is flow diagram illustrating the table-level classification process steps adopted by the system and method for text mining from one or more tables in accordance with an exemplary embodiment of the present invention;

FIG. 2b is flow diagram illustrating the cell-level classification process steps adopted by the system and method for text mining from one or more tables in accordance with an exemplary embodiment of the present invention;

FIG. 3 is diagram illustrating quad-directional LSTM;

FIG. 4 is a schematic system diagram of the present invention; and

FIG. 5 is flow diagram illustrating the process steps adopted by the system and method for text mining from one or more tables in accordance with a further embodiment of the present invention.

### DETAILED DESCRIPTION

The present invention may be utilised in the context of chemistry research and patent specifications, and it will be convenient to describe the invention in relation to that exemplary, but non-limiting, application. It will be appreciated that the present invention is not limited to that application and may for example, be applied in web-tables or, for instance scientific publications with tables. Advantageously, the present invention may utilise a web-table dataset which may be used for evaluating a cell-level classification task. Another application may be, for example evidence-based medicine where summary data related to clinical trial populations often appears in tables or in a systematic review paper where key data elements of the studies included in the review will often be summarised in a table. In addition, it will be appreciated that the present invention applies to other tables contained within, for example corporate annual reports.

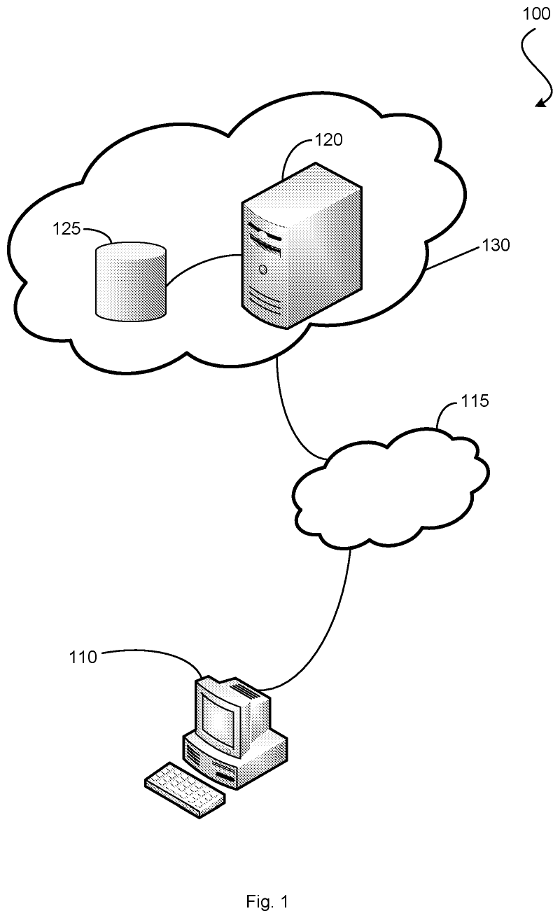

Referring to FIG. 1, there is shown a diagram of a system and method 100 for text mining from a table, with devices making up the system, in accordance with an exemplary embodiment of the present invention. The system 100 includes one or more servers 120 which include one or more databases 125 and one or more computing devices 110 (associated with a user for example) communicatively coupled to a cloud computing environment 130, “the cloud” and interconnected via a network 115 such as the internet or a mobile communications network.

Although “cloud” has many connotations, according to embodiments described herein, the term includes a set of network services that are capable of being used remotely over a network, and the method described herein may be implemented as a set of instructions stored in a memory and executed by a cloud computing platform. The software application may provide a service to one or more servers 120, or support other software applications provided by a third party servers. Examples of services include a website, a database, software as a service, or other web services. Computing devices 110 may include smartphones, tablets, laptop computers, desktop computers, server computers, among other forms of computer systems.

The transfer of information and/or data over the network 115 can be achieved using wired communications means or wireless communications means. It will be appreciated that embodiments of the invention may be realised over different networks, such as a MAN (metropolitan area network), WAN (wide area network) or LAN (local area network). Also, embodiments need not take place over a network, and the method steps could occur entirely on a client or server processing system.

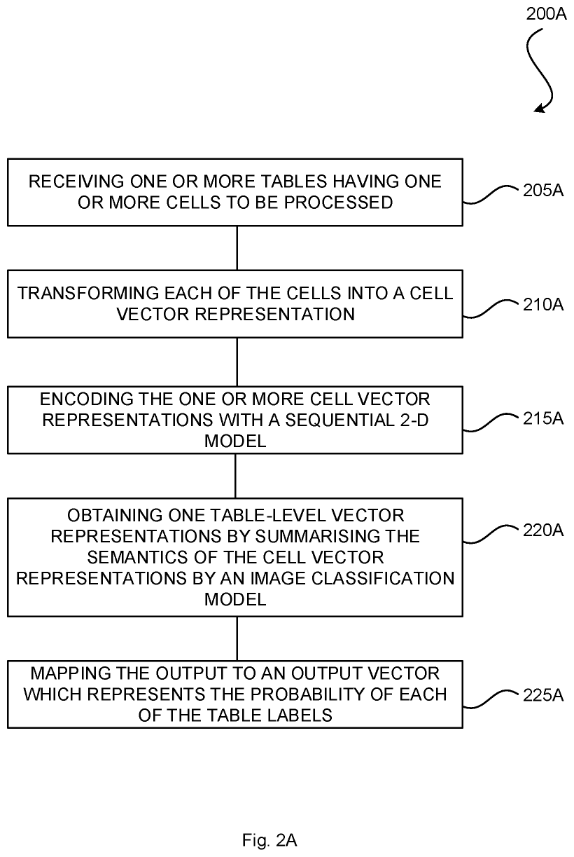

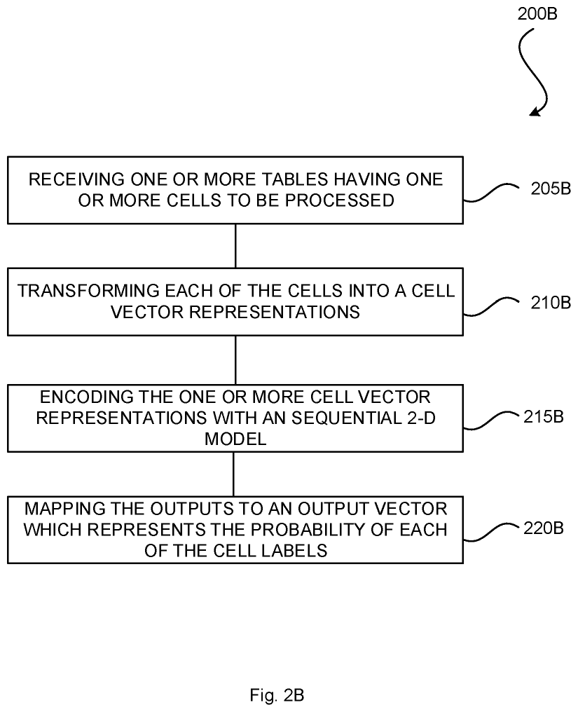

Referring now to FIG. 2a, there is shown a flowchart illustrating the process steps 200a for table-level classification adopted by the system and method for text mining from one or more tables in accordance with an exemplary embodiment of the present invention. The method begins at step 205a where a user associated with, for example the computing device 110 of FIG. 1 provides table data having one or more cells to be processed which are ultimately uploaded and received by the server 120 and stored in the database 125 in cloud 130 by way of, for example network connection 115. The table data may take any suitable form and may for example be tables within an overall document such as a patent specification and the tables may be provided in any number of formats including PDF, jpg, tiff and the like. Preferably, in the present invention the tables, where provided in image formats, are converted into plain text/xml format.

Control then moves to step 210a, where software residing on server 120 and database 125 in cloud 130 transforms each of the cells into cell vector representations. Control then moves to step 215a where the one or more cell vector representations are encoded with a sequential 2-D model. At step 220a one or more table-level vector representations are obtained by summarising the semantics of the cell vector representations by an image classification model. Control then moves to step 225 where the output of step 220a is mapped to an output vector which represents the probability of each of the table labels.

Referring now to FIG. 2b, there is shown a flowchart illustrating the process steps 200b for cell-level classification adopted by the system and method for text mining from one or more tables in accordance with an exemplarily embodiment of the present invention. The method begins at step 205b where a user associated with, for example the computing device 110 of FIG. 1 provides table data having one or more cells to be processed which are ultimately uploaded and received by the server 120 and stored in the database 125 in cloud 130 by way of, for example network connection 115. The table data may take any suitable form and may for example be tables within an overall document such as a patent specification and the tables may be provided in any number of formats including PDF, jpg, tiff and the like. Preferably, in the present invention the tables, where provided in image formats, are converted into plain text/xml format.

Control then moves to step 210b, where software residing on server 120 and database 125 in cloud 130 transforms each of the cells into cell vector representations. Control then moves to step 215b where the one or more cell vector representations are encoded with a sequential 2-D model. At step 220b, for each cell, the outputs of step 215b are then mapped to an output vector which represents the probability of each of the cell labels.

More generally, a user associated with, for example the computing device 110 of FIG. 1 may provide table data having one or more cells to be processed which are ultimately uploaded and received by the server 120 and stored in the database 125 in cloud 130 by way of, for example network connection 115. The table data may take any suitable form and may for example be tables within an overall document such as a patent specification and the tables may be provided in any number of formats including PDF, jpg, tiff and the like. Preferably, in the present invention the tables, where provided in image formats, are converted into plain text/xml format.

Software residing on server 120 and database 125 in cloud 130 classifies each of the one or more tables received by way of table level classification and assigns a label to each table. This step attempts to predict a content type of a complete table. In an alternative embodiment, this step may be omitted. There may be two types of table-level classification which can apply, table layout classification and/or table semantic classification.

Table layout classification predicts how a table is organised. For example, web tables maybe classified into 3 major categories (relational, entity and matrix) based on their contents as shown in Table 1 below.

| | Lake | Area |
|---|---|---|
| 1 | Windermere | 5.69 sq mi (14.7 km²) |
| 2 | Kielder Reservoir | 3.86 sq mi (10.0 km²) |
| 3 | Ullswater | 3.44 sq mi (8.9 km²) |
| 4 | Bassenthwaite | 2.06 sq mi (5.3 km²) |
| | Lake | |
| 5 | Derwent Water | 2.06 sq mi (5.3 km²) |

(a) Relational Table

| Government | |
|---|---|
| Type | Mayor- |
| | Council |
| Body | New York |
| | City Council |
| Mayor | Bill de Blasio (D) |
| Area | |
| Total | 468.9 sq mi |
| | (1,214 km²) |
| Land | 304.8 sq mi |
| | (789 km²) |
| Water | 164.1 sq mi |
| | (425 km²) |
| Metro | 13,318 sq mi |
| | (34,490 km²) |
| Elevation | 33 ft (10 m) |

(c) Entity Table

| | Right- | | |
|---|---|---|---|
| | handed | Left-handed | Total |
| Males | 43 | 9 | 52 |
| Females | 44 | 4 | 48 |
| Totals | 87 | 13 | 100 |

(b) Matrix Table

Table 1a, 1b, 1c: Examples of web tables with different layouts.

For relational tables as shown in Table 1a, they can be further categorised by their orientations (either horizontally or vertically orientated). The table layout can then be used as a feature for subsequent information extraction tasks on these tables.

The other type of table-level classification that may be carried out is table semantic classification, which predicts the label of the tables based on their content type. For example, web tables may contain data of a wide range of objects, such as location, person and events. Understanding the content type of the tables can assist in locating tables with the most relevant information and improve information extraction techniques by specifically focusing on each category of the table. In the present invention, in the case of tables in chemical patent specifications, the data can contain radically different types of data such as spectroscopic, pharmacological and reaction related data as shown in Table 2a and 2b.

| Ex. | Structure | Purification, Physical properties |
|---|---|---|
| 3 | 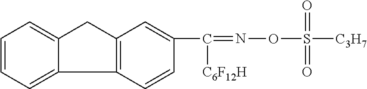 | Recrystallization from 2-propanol ¹H-NMR and ¹⁹F-NMR (CDCl₃) δ [ppm]: 1.10 (t, 3H), 1.87-1.98 (m, 2H), 3.39 (t, 2H), 3.98 (s, 2H), 6.05 (tt, 1 H), 7.33-7.43 (m, 3H), 7.54-7.62 (m, 2H), 7.84 (d, 1 H), 7.88 (d, 1H), −137.40 (d, 2F), −129.74 (s, 2F), −123.80 (s, 2F), −121.43 (s, 2F), −120.55 (s, 2F), |
| | | −109.83 (s, 2F), tentatively assigned as E- |
| | | configuration |
| | | White solid, mp: 66-68° C. |
| 4 | 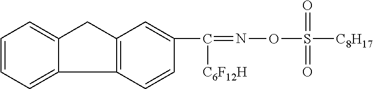 | Recrystallization from 2-propanol ¹H-NMR and ¹⁹F-NMR (CDCl₃). δ [ppm]: 0.89 (t, 3H), 1.20-1.50 (m, 10H), 1.83-1.96 (m, 2H), 3.40 (t, 2H), 3.98 (s, 2H), 6.05 (tt, 1H), 7.33-7.48 (m, 3H), 7.53-7.63 (m, 2H), 7.88 (d, 1H), 7.88 (d, 1H), −137.47 (d, 2F), −129.75 (s, 2F), −123.81 (s, 2F), −121.45 (s, |
| | | 2F), −120.02 (s, 2F), −109.81 (s, 2F), tentatively |
| | | assigned as E-configuration |
| | | White solid, mp: 78-79° C. |

(a) Example of a self-contained table describing spectroscopic data of compounds. Columns in this table are organized by data format (i.e. images, texts)

Table 2. Specific activity of sialidases (units per mg).

| Sialidase | Specific activity |
|---|---|
| AR-NEU2 | 8 |
| AR-AvCD | 937 |
| C. perfringens | 333 |
| A. ureafaciens | 82 |

(b) Example of a pharmacological table containing only pointers to contents the body of the patent documents. Columns in this table is organized by data type (i.e. different activity range).

### Table 2a and 2b

However, not all tables are considered relevant for researchers. This is particularly the case in patent specifications but more generally, where there are many tables in a document, some tables may be more relevant to a given information need than other tables and the characterisation can be useful to assist prioritising them. In view of the size and number of tables in chemical patent specifications (typically much larger than in web pages, for example) the present invention provides a system and method which can categorise the tables automatically to help reduce effort for extracting data from the tables.

The data may be further classified in that the one or more cells in the one or more tables are classified by cell-level classification to provide one or more classified cells and to assign a label to each cell.

Cell-level classification requires the system to make a decision on the content type of the table at a finer level. A finer level than table level classification is desirable since table level classification is at a broader category applicable to the whole table, whereas cell-level allows for capturing the detail of the information in the table. In this step, where possible, a label is applied to every cell in the table to indicate the type of information that is in the table. For example, in a structural table it is preferable to determine a structural label (e.g. header, sub-header, data, image, etc) for each cell based on its content. For example, as shown in Tables 3a to 3e.

Summary of Test results of compounds 1-4, and comparison to SAHA results.

| MEL Differentiation | | | | | |
|---|---|---|---|---|---|
| Com- | | | | HDAC Inhibition | |
| pound | Range | Optimum | % B+ | Range | ID50 |
| 1 | 0.1 to 50 μM | 200 nM | 44% | 0.0001 to 100 μM | 1 nM |
| 2 | 0.2 to 12.5 μM | 800 nM | 27% | | TBT |
| 3 | 0.1 to 50 μM | 400 nM | 16% | 0.01 to 100 μM | 100 nM |
| 4 | 0.01 to 50 μM | 40 nM | 8% | 0.01 to 100 μM | <10 nM |
| SAHA | | 2500 nM | 68% | 0.01 to 100 μM | 1000 nM |

% B+ = Percentage Benzidine positive cells
(a) Example of a pharmacological table in chemical patents.

| CAPTION | Placeholder | Placeholder | Placeholder | Placeholder | Placeholder |
|---|---|---|---|---|---|
| Blank | Header_Col | < Merged | < Merged | Header_Col | < Merged |
| Header_Row | SubHeader_ | SubHeader_ | SubHeader_ | SubHeader_ | SubHeader_ |
| | Col | Col | Col | Col | Col |
| Header_Row | Data | Data | Data | Data | Data |
| Header_Row | Data | Data | Data | Blank | Data |
| Header_Row | Data | Data | Data | Data | Data |
| Header_Row | Data | Data | Data | Data | Data |
| Header_Row | Blank | Data | Data | Data | Data |
| Footer | < Merged | < Merged | < Merged | < Merged | < Merged |

(b) Example of the cell-level annotation of the table above.

| | Lake | Area |
|---|---|---|
| 1 | Windermere | 5.69 sq mi (14.7 km²) |
| 2 | Kielder Reservoir | 3.86 sq mi (10.0 km²) |
| 3 | Ullswater | 3.44 sq mi (8.9 km²) |
| 4 | Bassenthwaite | 2.06 sq mi (5.3 km²) |
| | Lake | |
| 5 | Derwent Water | 2.06 sq mi (5.3 km²) |
| Blank | H.Col | H.Col |
| ID | Data | Data |
| ID | Data | Data |
| ID | Data | Data |
| ID | Data | Data |
| ID | Data | Data |

(c) Web Table Relational Table

| | Right- | Left- | |
|---|---|---|---|
| | handed | handed | Total |
| Males | 43 | 9 | 52 |
| Females | 44 | 4 | 48 |
| Totals | 87 | 13 | 100 |
| Blank | H.Col | H.Col | H.Col |
| H Row | Data | Data | Data |
| H Row | Data | Data | Data |
| H Row | Data | Data | Data |

(d) Web Table Matrix Table

| Government | |
|---|---|
| Type | Mayor-Council |
| body | New York City Council |
| Mayor | Bill de Blasio (D) |
| Area | |
| Total | 468.9 sq mi (1,214 km²) |
| Land | 304.8 sq mi (789 km²) |
| Water | 164.1 sq mi (425 km²) |
| Metro | 13,318 sq mi (34.490 km²) |
| Elevation | 33 ft (10 m) |
| H_Row | ←Merged |
| ↑SH_Row | Data |
| ↑SH_Row | Data |
| ↑SH_Row | Data |
| ↑SH_Row | Data |
| ↑SH_Row | Data |
| ↑SH_Row | Data |
| ↑SH_Row | Data |
| H_Row | Data |

(e) Web Table Entity Table

Tables 3a to 3e: Examples of cell-level annotations on chemical patent tables and web tables.

The structural label of table cells can then be used as features for other tasks such as table processing, table layout classification and cell-level relation extraction. Cell-level relation extraction refers to associating information between cells. For example, linking at a specific “range” data value in Table 3a, above, the relevant compound, such as relating 0.2 to 12.5 4M “range” to compound “2”. This also includes, for example the relationship between the cells labelled “<Merged” and the primary cell it is merged into in Table 3b, above, or the relationship between the data values in a column and the column header (e.g. the relationship between the cell containing “27%” and “% B+”).

Each of the one or more classified cells in the tables may be pre-processed to provide one or more pre-processed classified cells. The tables may then be transformed into a suitable format for the next stage of analysis in which a pre-processing step called tokenization may be provided on the content of each cell. In an alternative embodiment, the pre-processing of classified cells may be omitted in that the inputs need not necessarily be classified. In this arrangement strings are split into substrings corresponding to words, symbols or punctuation marks. Pre-processing data sets may be provided and in the chemical domain these may include, for example, ChemTables and a tokenizer such as OSCAR4 may be used. It will be appreciated that other chemical table data sets may be utilised as required but it will also be appreciated that the invention is not limited to the specific data set that is being utilised. Other tokenizers optimised for chemical documents may be utilised, for example ChemTok, NBICGeneChemTokenizer but it has been found that OSCAR4 is an optimal choice among them.

As will be appreciated by a person skilled in the art, any suitable chemical tokenizer may be utilised or one could be built. For documents that aren't related to chemistry, for example any open sourced tokenizer may be utilised e.g. OpenNLP, CoreNLP, NLTK, spaCy Tokenizer and the like.

It will be appreciated that tokenization depends on the structure of the strings in the text and the objective is to find word boundaries. These word boundaries may vary in different domains or type of texts. For example, in biological publications, tokenizers that can appropriately deal with DNA strings would be required, whereas for clinical texts for example, tokenizers might tokenizer things such as blood pressure strings which may take the form of numbers such as “ 120/80”.

OSCAR4 is an example of a tokenization component of an open source tool kit optimised for chemical applications and focuses on named entity recognition of chemical entities such as chemical names. It will be appreciated that depending on the application a different type of tokenizer may be used. Advantageously, choosing a tokenizer specially defined for chemistry applications helps to improve performance. An example ChemTables dataset is shown in Table 4a, 4b and 4c.

TABLE 2
Specific activity of sialidases (units per mg).

| Sialidase | Specific activity |
|---|---|
| AR-NEU2 | 8 |
| AR-AvCD | 937 |
| C. perfringens | 333 |
| A. ureafaciens | 82 |

(a) An example of chemical patent table in PDF format.

TABLE 2

| ‘Example | ‘Compound | ‘Activity Range | Activity Range |
|---|---|---|---|
| Number’ | Number’ | (IC50)’ | (EC50)’ |
| ‘1’ | ‘1’ | ‘D’ | ‘B’ |
| ‘2’ | ‘2’ | ‘D’ | ‘B’ |
| ‘3’ | ‘3’ | ‘D’ | ‘C’ |
| ‘4’ | ‘4’ | ‘D’ | ‘B’ |

(b) An example of chemical patent table in the Excel file

TABLE 2

[‘Example’, ‘Number’], [‘Compound’, ‘Number’], · · ·
· · · [‘Activity’ ‘Range’, ‘(‘, ‘IC50’, ’)’], [‘Activity’
‘Range’, ‘(‘, ‘EC50’, ’)’] ]
[‘1’], [‘1’], [‘D’], [‘B’]]
[‘2’], [‘2’], [‘D’], [‘B’]
[‘3’], [‘3’], [‘D’], [‘C’]
[‘4’], [‘4’], [‘D’], [‘B’]

(c) An example of 2-D list resulting from pre-processing.

Table 4: Example of pre-processing data set.

In the examples above in Table 4a, the data is extracted using any suitable arrangement from a patent document which typically would be in PDF format or the like. The extracted tables from the patent may be stored in a suitable format such as in an Excel table and example of which is shown in Table 4b. In an embodiment, there may be provided a pre-processing step where the tables are read from the Excel table and then the tokenizer (such as OSCAR4 or the like) is applied on the content of each cell to split it into individual words which results in a 2-dimensional list as shown in Table 4c.

Each of the one or more pre-processed cells may then transformed into one or more cell vector representations. This is in order to attempt to classify the tables at cell or table level based on the type of content and to achieve this the model transforms the text content of each cell into a vector representation which allows mathematical modelling and comparison of that content across tables.

The one or more cells vector representations may be encoded by way of an artificial recurrent neural network (RNN) which preferably takes the form of a Quad-directional long-short term memory network (Q-LSTM). The present invention may be utilised with diagonal LSTM but preferably Q-LSTM is proposed. For example, for image completion and its receptive field it is restricted so it cannot see what it needs to predict during training. In additional, the inputs of the two tasks are different (images versus tables) which means that diagonal LSTM can be applied to table classification but is preferably modified to be quad-directional LSTM for optimal performance.

It will be appreciated that other RNN models may also be utilised, but in a preferred embodiment the present invention utilises Q-LSTM.

In essence, the present invention captures dependencies between cells using a sequential model. That sequential model is preferably quad-directional LSTM but may be, for example diagonal LSTM or any sequential model that works on two-dimensional data. Essentially, the present invention is providing a sequential model adapted to capture table semantics.

Advantageously, the present invention leverages semantic information by embedding table cells with pre-trained language models while respecting the structural information of tables and sequential relation between cells by using the quad-directional diagonal-LSTM.

Since semantic types of data in cells are usually dependent on the header cells which can be far away from it to capture such long-term dependency between cells the one or more cell vector representations are encoded by way of quad-directional LSTM. This will be further described below.

Wherein an embodiment, after the encoding steps, one or more table-level vector representations may be obtained by summarising the sematic of the cell vector representations associated with the table by image classification. For example, image classification may be carried out by way of ResNet18 which is a widely used model for image classification to obtain table-level vector representation which will be further described below.

An output layer may then map the output to an output vector which represents the probability of each of the table labels and cell labels and providing a label data set.

The present invention has an approach to analysing table semantics based on adapting models proposed for image processing to table processing. In image processing, each pixel can be represented as a vector of uniform size. In the context of tables, the text in each cell can be a variable length and is significantly more complex in content. Therefore, the present invention develops an approach to represent the content of the cells in a vector of uniform size and an embedding step carries this out. In one embodiment a word representation may be provided where a combination of pre-trained word vector and character-level word representation is provided as input to the embedder. Word embeddings are dense vectors which represent word semantics in a relatively low dimension space. Pre-trained word embeddings are usually learned by aggregating word-word co-occurrence statistics from a large amount of text data. This can be carried out in any suitable manner and for example, pre-trained word vectors such as that found in via existing unsupervised learning algorithms for obtaining vector representations (such as GLoVe for example). It will be appreciated that it may be carried out in any other suitable manner, such as by way of Word2vec and Continuous-Bag-of-Words (CBOW) for pre-trained word vectors. Long-text transformers may be directly used as the encoder and are preferably for example, where there are large-tables (long inputs). However, as will be appreciated by a person skilled in the art, contextualised word representations generated by ELMo or BERT, or the like, can also be potentially used. Essentially, any suitable method for inferring word embeddings from a background text collection can be adopted.

Other data sets containing chemical tables could be utilised such as ChemPatent. In the present invention, in addition to pre-trained word vectors, the character-level word representation may be used to capture the morphological information within words. For example, for character-level word representation, a convolutional neural network (CNN)-based approach may be utilised with a filter size of, for example, 3. In addition, an architecture may be utilised such as bi-directional LSTM with word representation and character-level word representation. Bi-directional LSTM is a variant of an RNN which runs in both forward and backward directions over a sequence and in this case a sequence of words or characters. It will be appreciated that the “directionality” does not have to be directional or bi-directional. For example, “simpler” models may be utilised that just capture “n-grams” (i.e. substrings of the length n) which, for example was commonly utilised in pre-neural feature engineering-based machine learning methods.

The present invention preferably utilises quad-directional LSTM. Quad-directional LSTM extends the concept of diagonal LSTM (which itself is an extension of standard LSTM which runs in diagonal or 2-dimensional input). It has the advantage of capturing long term dependency compared to say, conventional RNN. Instead of taking a one-dimensional sequence as input, diagonal LSTM takes inputs from two directions in a two-dimensional plane. The present invention extends the concept of diagonal LSTM from images to tables and identifies an analogy between tables and images in that both the tables and images are two-dimensional structured data. The present invention adapts this model to the context of tables, enabling to capture long-term dependencies between tables cells without loss of structural information.

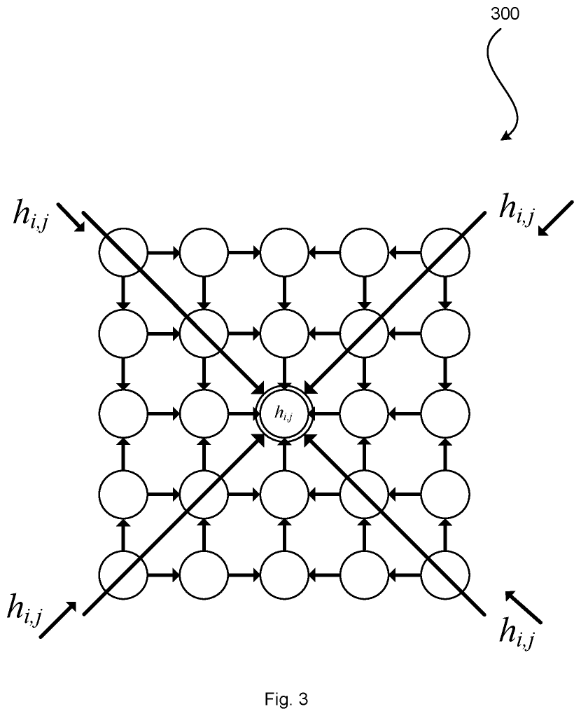

In the present invention, quad-directional LSTM is applied by adapting a two-dimensional LSTM network structure but applying it along four-diagonal directions. As shown in FIG. 3 to allow the model 300 to capture dependencies along the diagonal, Q-LSTM takes hidden and cell states from both the cell to the left and to the top of the current position as hi-1 and ci-1.

[oᵢ, fᵢ, iᵢ, gᵢ]=σ(K^{hs}\textcircled{*}hᵢ₋₁+K^{is}\textcircled{*}xᵢ) (1)

cᵢ=fᵢ⊙K^{cc}\textcircled{*}cᵢ₋₁iᵢ⊙gᵢ (2)

hᵢ=oᵢ⊙tan h(cᵢ) (3)

In Q-LSTM cell, a 2×1 convolution is applied to combine previous hidden and cell states from both horizontal and vertical direction. As shown in Equation 1 and 2, the weight for hidden-to-state and cell-to-state components are denoted as Khs and Kis respectively. Since input at the current position is also needed when calculating the states for gates, a 1×1 convolution is applied which samples the input vector to the same dimension as hidden size of the Q-LSTM. Then, as shown in Equation 2 and 3, the current cell state and hidden state can be calculated in the same manner as in conventional LSTM. A residual connection is added from the cell-level embedder to Q-LSTM by concatenating the output of these two layers.

Weighted sum is used to combine the hidden state generated by Diagonal LSTM running in 4 different directions.

H=Σ_{d∈D}W_{d}H_{d} (4)

As shown in Equation 4, D={ [symbol] , [symbol] , [symbol] , [symbol] } denotes the set of 4 directions, Hd and Wd denotes the hidden states generated by Diagonal LSTM and weight matrix for d direction, respectively.

In an embodiment, one or more table vector representations may be obtained by summarising the semantic of the cell vector representations associated with the table by way of image classification and this may be for example carried out by using ResNet18 as a decoder. Advantageously, ResNet18 is a powerful decoder which summarises the semantic information in the hidden state of each cell (even those Q-LSTM can capture sequential information between cells).

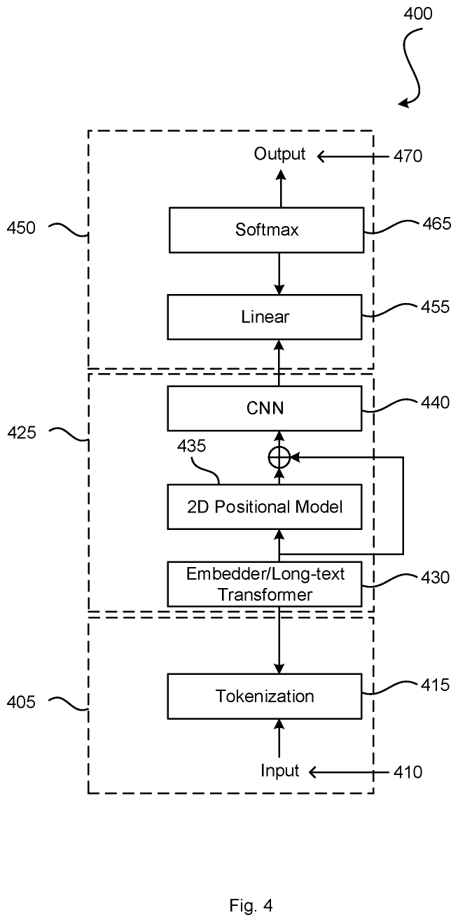

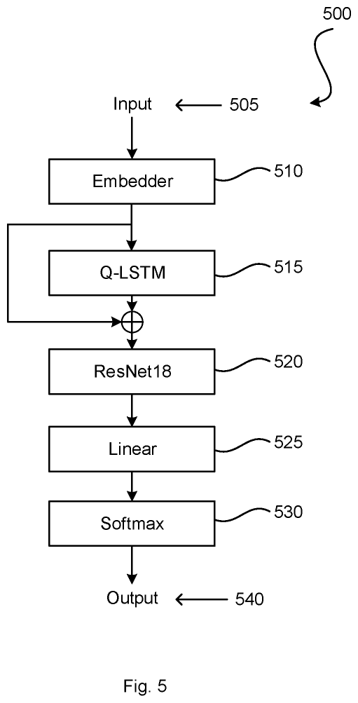

FIG. 4 is a system diagram of the present invention, illustrating a pre-processing component 405 with an encode-decode component 425 and an output component 450. As will be further described with reference to FIG. 5, the pre-processing component includes an input 410 and a tokenization step 415. Connected to the pre-processing component 405 is a encode-decode component 425. This component includes embedding/long-text transforming steps 430, applying a 2D positional model 435, and a convolutional neural network (CNN) 440. The output component 450 contains linear component step 455 output to a softmax function 465 which is output to the overall output 470. Pre-processing may be carried out at the tokenization step 425, where for an LSTM-based embedder tokenization is by way of OSCAR4 or the like. Whereas, for a transformer embedder, tokenization may be by way of a tokenizer associated with the model (where each transformer includes its own sub-word tokenizer) and linearization. The 2D positional model step 435 receives feedback from the embedder/long-text transformer step 430 to the convolutional neural network (CNN) 440.

As shown in FIG. 5, the present invention may combine Q-LSTM and ResNet18 by using Q-LSTM as a second layer encoder on top of a cell-level embedder. ResNet18 is an 18-layer residual network proposed for image classification, where ResNet18 can be used as the decoder which summarises the semantic representation of the table contents generated by the earlier steps of the process. As will be appreciated by a person skilled in the art, any image classification method may be substituted and for example may include but is not limited to VGG, DenseNet, Inception, or the like.

In an embodiment, as described with reference to FIG. 5, a 3-layer combined architecture is provided, and cell content is summarised using the cell-level embedder and sequential information is captured between cells by Q-LSTM and summarised at table level by ResNet18. At steps 505 to 510 a vector representation is obtained for each cell. Control then moves to step 515 where Q-LSTM is used to map cell vectors into another vector space in which the information of neighbouring cells are incorporated. Control then moves to step 520 in which ResNet18 is utilised to summarise cell-level semantics to table level. Control then moves to step 525 through to 540, where table level semantics are mapped to a probabilistic distribution over all table categories and the most likely category is then selected.

In a further embodiment, as described with reference to FIG. 5 a transformer-based language model which is pretrained on a large amount of unannotated data is provided together with Q-LSTM and ResNet18. A transformer based language model may be BERT, which may generate contextualized word representations by combining its internal states for use in NLP tasks to improve performance. A technique may be utilised such as BERT for capturing table semantics. In practice, BERT is pre-trained on one-dimensional texts only which means tables being two-dimensional structure texts cannot be used as input to these language models.

As shown in Table 5, below, the input of Table-BERT is prepared using linearization (e.g. concatenating table cells from left to right, top to bottom) to produce improved results in classifying tables in chemical patents.

TABLE 1
Affinities to Heparin

| Protein | Kd nM (ref) |
|---|---|
| PF4 | 27 (44) |
| IL-8 | <5 (43) |
| ATIII | 11 (42) |
| ApoE | 620 (45) |

Key:
[EOS] = End of sentence
[SEP] = End of paragraph
[CLS] = Token used to pool the representation of the table
Bold Underline = Caption = “-------------------”
Underline = Row headers = “.................................”
Italics = Column headers = “._._._._._._._._._._._._._._”
Linearization
[CLS] Table, 1,., Affinities, to, Heparin, [EOS] Protein, Kd, nM, (, ref,) [EOS] PF4, 27, (, 44,), [EOS] IL, −, 8, <, 5, (, 43, ) [EOS] ATIII, 11, (, 42,) [EOS] ApoE, 620, (, 45,) [SEP]
Table 5: An example of linearization pre-processing approach in Table-BERT

In a further embodiment, as described with reference to FIG. 5, the present invention may utilise Q-LSTM's ability in capturing structural relations between cells. In this approach, the combined model is trained (the combined model being Q-LSTM plus ResNet18) by summing their logits generated by the linear layer 525. In particular, FIG. 5 shows the architecture which leverages the advantages Q-LSTM in addition to ResNet18. At step 505, input is received and pre-processed by tokenization and control then moves to step 510, where: 1. tokenized input is fed into a Bi-LSTM-CNN embedder to obtain cell-level vector representation; or 2. the tokenized input is linearized and fed into a transformer, before control moves to step 515, where sequential relationships are captured between cells by applying Q-LSTM on cell-level vectors generated from step 510. At step 520, ResNet18 is applied on the output of step 515 to summarise cell-level vectors and generate one single table level vector in which table semantics are embedded. Control then moves to step 525 in which a linear layer is applied to project table-level vectors generated by step 520 to logits which are of the same size as the number of table categories in a dataset. Control then moves to step 530, where a softmax function is applied on the sumlogits generated at step 525 to obtain a probability distribution over all table categories in a dataset. Control then moves to step 535 where a category with the highest probability score in step 530 is selected as the predicted label.

While the invention has been described in conjunction with a limited number of embodiments, it will be appreciated by those skilled in the art that many alternatives, modifications and variations in light of the foregoing description are possible. Accordingly, the present invention is intended to embrace all such alternatives, modifications and variations as may fall within the spirit and scope of the invention as disclosed.

1. A method for text mining from one or more tables, the method including the steps of:
(a) receiving one or more tables, the tables having one or more table labels, and one or more cells to be processed;
(b) transforming each of the cells into cell vector representations;
(c) encoding the one or more cell vector representations with a sequential 2-D model;
(d) obtaining one or more table-level vector representations by summarising the semantics of the cell vector representations by an image classification model; and
(e) mapping the output of step (d) to an output vector which represents the probability of each of the table labels.

2. The method of claim 1, wherein the sequential 2D model includes one or more quad-directional long-short term memory network.

3. The method of claim 1, wherein the sequential 2D model is Q-LSTM.

4. The method of claim 1, further including the step of: applying a machine learning paradigm to train a model from a labelled data set.

5. The method of claim 1, wherein a long-text transformer is provided as the encoder.

6. The method of claim 1 wherein a combination of pre-trained word vectors and character-level word representation is provided as input to the encoder.

7. The method of claim 6, wherein the pre-trained word vectors are provided by an unsupervised learning algorithm for obtaining vector representations.

8. The method of claim 1 wherein, the encoder is pre-trained with an in-domain dataset.

9. The method of claim 7, wherein the algorithm is selected from one or more of a long-text transformer encoder, GLoVe, Word2vec, Continuous-Bag-of-Words (CBOW), ELMo or BERT. (Original) The method of claim 1, wherein the table level classification includes a table layout classification.

11. The method of claim 1, wherein the table level classification includes a table semantic classification.

12. The method of claim 1, wherein the method includes a step of pre-processing the one or more classified cells into each of the one or more tables to provide one or more pre-processed classified cells.

13. The method of claim 1, wherein the pre-processing is tokenisation by way of one or more of OSCAR4, ChemTok, NBICGeneChemTokenizer, OpenNLP, CoreNLP, NLTK, spaCy Tokenizer and the like.

14. The method of claim 1, wherein the image classification is by way of a convolutional neural network such as one or more of esNet18, VGG, DenseNet or Inception.

15. The method of claim 1, wherein the step of transforming each of the cells into cell vector representations includes utilising a long-text transformer or an LSTM-based embedder.

16. The method of claim 1, wherein the method includes the step utilising a transformer based language model, and generating contextualized word representations by combining the internal states of the model for use in Natural Language Processing (NLP) tasks.

17. The method of claim 16, wherein the language model is one or more of a long-text transformer encoder, BERT, ELMo, XLNet or Roberta.

18. The method of claim 17, wherein the language model BERT is modified to accept tables.

19. A method for text mining from one or more tables, the method including the steps of:
(a) receiving one or more tables, the tables having one or more cell labels, and one or more cells to be processed;
(b) transforming each of the cells into cell vector representations;
(c) encoding the one or more cell vector representations with a sequential 2-D model; and
(d) for each cell, mapping the outputs of step (c) to an output vector which represents the probability of each of the cell labels.

20. The method of claim 16, wherein the sequential 2D model includes one or more quad-directional long-short term memory network.

21. The method of claim 16, wherein the sequential 2D model is Q-LSTM.

22. The method of claim 16, further including the step of: applying a machine learning paradigm to train a model from a labelled data set.
